(19) United States
(12) Patent Application Publication
(10) Pub. No.: US 2009/0005325 A1
(43) Pub. Date: Jan. 1, 2009

(54) Ketolide Derivatives as Antibacterial Agents

(76) Inventors: Biswajit Bas, Gurgaon (IN); Anjan Chakrabarti, Gurgaon (IN); Ramadass Venkataramanan, New Delhi (IN); Atul Kashinath Hajare, Ahmednagar (IN); Anish Bandyopadhyay, Gurgaon (IN); Rita Katoch, Chandigarh (IN); Gobind Singh Kapkoti, Navi Mumbai (IN); Yogash Baban Surase, Nasik (IN); Mohammad Salman, Princeton, NJ (US); Dilip J. Upadhyay, Thane (IN)

Correspondence Address: RANBAXY INC., 600 COLLEGE ROAD EAST, SUITE 2100, PRINCETON, NJ, 08540, US

(21) Appl. No.: 12/094,839

(22) PCT Filed: Nov. 21, 2006
(86) PCT No.: PCT/IB06/03298

§ 371 Date: Sep. 10, 2008

(30) Foreign Application Priority Data
Nov. 23, 2005 (IN) 3138/DEL/2005

(51) Int. Cl. A61K 31/7048; A61P 31/12; C07H 17/08

(52) U.S. Cl. 514/29; 536/7.4

(57) ABSTRACT

Provided herein are ketolide derivatives, which can be used as antibacterial agents. Compounds described herein can be used for treating or preventing conditions caused by or contributed to by gram positive, gram negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Enterobactericeae, Propionibacterium acnes or any combination thereof. Also provided are processes for preparation of compounds described herein, pharmaceutical compositions thereof, and methods of treating bacterial infections.

### FIELD OF INVENTION

Provided herein are ketolide derivatives, which can be used as antibacterial agents. Compounds described herein can be used for treating or preventing conditions caused by or contributed to by gram positive, gram negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Enterobactericeae, Propionibacterium acnes or any combination thereof. Also provided are processes for preparation of compounds described herein, pharmaceutical compositions thereof, and methods of treating bacterial infections.

### BACKGROUND OF THE INVENTION

First generation macrolides erythromycin A and early derivatives are characterized by bacteriostatic or bactericidal activity for most gram-positive bacteria, atypical pathogens, and many community-acquired respiratory infections and in patients with penicillin allergy. However, erythromycin A causes numerous drug-drug interactions, has relatively poor absorption, poor local tolerance, loses its antibacterial activity under acidic conditions by degradation and the degraded products are known to be responsible for undesired side effects (Itoh, Z et al., Am. J. Physiol, 1984, 247:688; Omura, S et al., J. Med. Chem., 1987, 30:1943). Various erythromycin A derivatives have been prepared to overcome the acid instability and other problems associated with it.

Roxithromycin, clarithromycin and azithromycin were developed to address the limitation of erythromycin A. Both clarithromycin and azithromycin were found to be important drugs in the treatment and prophylaxis of atypical mycobacterial infections in patients with HIV.

Macrolides were found to be effective drugs in the treatment of many respiratory tract infections. However, increasing resistance among S. pneumoniae has prompted the search for new compounds that retain favorable safety profiles, retain a spectrum of activity and are confined to respiratory pathogens. Consequently, numerous investigators have prepared chemical derivatives of erythromycin A in an attempt to obtain analogs having modified or improved profiles of antibiotic activity. Ketolides exhibit greater efficacy and safety, have broader spectrum of activities, and are particularly effective against resistant pathogens; hence, ketolides have been developed as next generation macrolides.

U.S. Pat. No. 5,635,485 discloses erythromycin compounds that are reportedly useful in the treatment of bacterial infections in warm-blooded animals. U.S. Pat. No. 5,866,549 discloses novel semi-synthetic macrolides reportedly having antibacterial activity, as well as 6-O-substituted erythromycin ketolide derivatives and a method of treating bacterial infections. U.S. Pat. Nos. 6,458,771 and 6,399,582 and PCT Publication Nos. WO 00/62783 and WO 00/44761 disclose ketolide antibacterials that are reportedly useful in treating bacterial and protozoal infections and in treating other conditions involving gastric motility. U.S. Pat. No. 5,747,467 discloses erythromycin and novel antibacterial composition and a method of treating bacterial infection in warm-blooded animals.

U.S. Pat. No. 6,433,151 discloses erythromycin derivatives and their use as medicament for treating infections caused by particular gram-positive bacteria, namely Haemophilus influenzae, and Morraxalla spp. U.S. Pat. No. 6,472,372 discloses 6-O-carbamoyl ketolide antibacterials and methods of treating bacterial infections. U.S. patent application Nos. 2002/0115621 and 2003/0013665 disclose macrolide compounds that are useful as antibacterial and antiprotozoal agents in mammals, including man, as well in fish and birds. Other ketolide compounds have also been reported. A. Denis and A. Bonnefoy, Drugs of the Future, 26(10):975-84 (2001), Champney W. S., et al., Current Microbiology, 42:203-10 (2001).

However, there remains a need for novel ketolide derivatives, which can be used as antibacterial agents on a wide variety of gram positive, gram negative or anaerobic bacteria.

### SUMMARY OF THE INVENTION

Provided herein are ketolide derivatives, which can be used in the treatment or prevention of bacterial infection, and processes for the synthesis of such compounds.

Pharmaceutically acceptable salts, pharmaceutically acceptable solvates, enantiomers, diastereomers, polymorphs of these compounds having same type of activity are also provided.

Also provided are pharmaceutical compositions containing the described compounds together with one or more pharmaceutically acceptable carriers, excipients or diluents, which can be used for the treatment of bacterial infection.

Thus in one aspect, provided herein are compounds having the structure of Formula I,

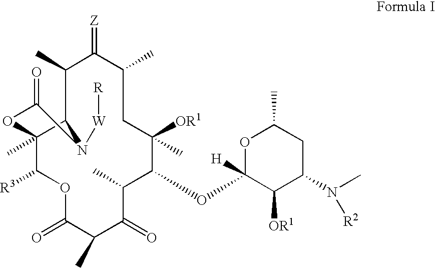

pharmaceutically acceptable salts, pharmaceutically acceptable solvates, enantiomers, diastereomers or polymorphs thereof, wherein

R¹ can be hydrogen or a hydroxyl protecting group;

R² can be C₂-C₆ alkyl, alkenyl, alkynyl, cycloalkyl, aryl, heterocycle, aralkyl, (heterocycle)alkyl or COR⁴, wherein
R⁴ can be hydrogen, alkyl or aralkyl;

W can be (CH₂)_{q}, CR⁵R⁶, NR⁵ or SO₂, wherein
q can be an integer of from 2 to 10;
R⁵ and R⁶ can be independently hydrogen or alkyl;
(CH₂)_{q} can be optionally interrupted by one or more of unsaturated bonds, oxygen, sulfur, CO, CS, SO₂, NR₁ or combination thereof; or
one or more hydrogen atoms of (CH₂)_{q} group can be optionally replaced by halogen, alkyl, hydroxyl or alkoxy; wherein
R₁ can be hydrogen, alkyl, alkenyl, alkynyl, COR₂ or (CH₂)ₘR₂;
R₂ can be alkyl, alkenyl, alkynyl, aryl or heterocycle;
R can be R₆NCOR₇, aryl or heterocycle;

R₆ and R₇ can be independently aryl or heterocycle;
R³ can be alkyl, alkenyl or alkynyl;
R′ can be alkyl or —(CH₂)ᵣ—U; wherein

r can be an integer of from 1 to 4; and

U can be alkenyl or alkynyl; and
Z can be oxygen, sulfur or NOR⁴; wherein

R⁴ is the same as defined earlier.

These compounds can include one or more of the following features. For example, R¹ can be hydrogen; R² and R³ can be ethyl; W can be (CH₂)_{q}, wherein (CH₂)_{q} is the same as defined earlier; R′ can be methyl; Z can be oxygen; and R can be heterocycle. In another feature, R¹ can be hydrogen; R² and R³ can be ethyl; W can be (CH₂)_{q}, wherein (CH₂)_{q} is the same as defined earlier; R′ can be methyl; Z can be oxygen; and R can be R₆NCOR₇,

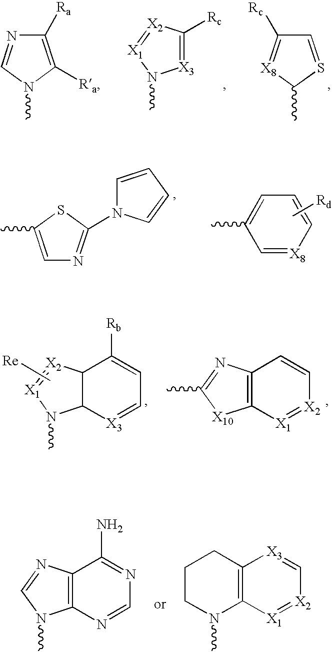

X₁-X₃ can be independently CH or N;
X₄-X₈ can be independently CH, CR₃ or N;
X₉ can be O,S,N,NH or CH; and
X₁₀ can be NH or S;
Rₐ can be optionally substituted thienyl, furyl, pyrazolyl, oxazolyl, tetrazolyl, imidazolyl, pyridinyl, fluoropyridinyl, chloropyridinyl, pyridinyl, pyrazinyl, pyrimidinyl, pyrimidinyl, quinolinyl, pyrrolo-pyridyl, pyrrolo-thiazolyl or phenyl wherein optional substituent(s) can be methyl, halogen, niethoxy, NR₄R₅, wherein

R₄ and R₅ can be independently hydrogen, methyl, isopropyl, cyclopropyl, or acetyl;
R′a can be hydrogen or furyl;
R_{b} can be hydrogen or NR₄R₅, wherein

R₄ and R₅ can be independently hydrogen, methyl, isopropyl, cyclopropyl, or acetyl;
R_{c} can be hydrogen, optionally substituted thienyl, furyl, pyrazolyl, pyrazinyl, pyridinyl, pyridinyl, pyrimidinyl, pyrrolyl, imidazolyl or phenyl wherein optional substituent(s) can be methyl, halogen, methoxy, NR₄R₅, wherein

R₄ and R₅ can be independently hydrogen, methyl, isopropyl, cyclopropyl, or acetyl;

R_{d} can be thienyl, pyrazolyl, imidazolyl, triazolyl, pyrrolyl or tetrahydrofuryl;

Rₑ can be (heterocyclyl)alkyl; and

R₆ and R₇ can be independently aryl or heterocyclyl.

In another aspect, provided herein are compounds selected from:

5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyridin-3-yl-methyl-benzimidazol-1-yl)-butyl)-imino)] erythromycin A,

5-O-(3′-N-desmethyl-3″-N-ethyl)desosaminyl-11,12-dideoxy-3-O-decladinosyl-6-O-methyl-3-oxo-12,11-[oxycarbonyl-((4-(4-pyridin-3-yl-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(imidazo[4,5-b]pyridin-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(3,4-dihydro-2H-quinolin-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-tetrazol-1-yl-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-3-yl-imidazol-1-yl)-butyl)-imino)] erythromycin A, 5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiophen-2-yl-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-2-yl-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyrazin-2-yl-thiazol-2-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyridin-3-yl-thiazol-2-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrazol-1-yl-pyridin-3-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(6-imidazol-1-yl-pyridin-3-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-amino phenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(6-pyrrol-1-yl)-pyridin-3-yl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrrol-1-yl-pyridin-3-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyrrol-1-yl-thiazol-5-yl)-butyl)-imino)] erythromycin A, 5-O-(3′-N-desmethyl-3′-N-ethyl)desosaminyl-11,12-dideoxy-3-O-decladinosyl-6-O-methyl-3-oxo-12,11- [oxycarbonyl-((4-([1,4′]-biimidazol-1′-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiaophen-2-yl-pyridin-3-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dimethyl-amino)-phenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-nicotinamido)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-pyrrol-1-yl-thiazol-5-yl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-benzamido)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-acetyl-amino)-phenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-isopropyl-amino)-phenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-fluoro-phenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-methoxy-phenyl)-imidazol-1-yl)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(benzthiazol-2-yl)-benzamido)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-isonicotinamido)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(5-methyl-pyridin-2-yl)-benzamido)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-imidazol-1-yl 1)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-5-yl)-imidazol-1-yl 1)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-4-yl)-imidazol-1-yl 1)-butyl)-imino)] erythromycin A,

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-pyrazol-1-yl 1)-butyl)-imino)] erythromycin A,

11,12-dideoxy-3-O- decladinosyl-5-O-(3′-N-desmethyl-3′-N-2-fluoroethyl)-6-O-methyl-3-oxo-12,11-[oxycarbonyl-(4-(4-(3-amino-phen-1-yl)-imidazol-1-yl)-butyl)-imino)] -erythromycin A,

11,12-dideoxy-3-O- decladinosyl-5-O-(3′-N-desmethyl-3′-N-2-fluoroethyl)-6-O-methyl-3-oxo-12,11-[oxycarbonyl-(4-(4-(2-amino-pyrimidin-5-yl)-imidazol-1-yl)-butyl)-imino)] -erythromycin A,

11,12-dideoxy-3-O- decladinosyl-5-O-(3′-N-desmethyl-3′-N-ethyl)-6-O-methyl-3-oxo-12,11-[oxycarbonyl-(4-(5-Nitro-indol-1-yl)-butyl)-imino] -erythromycin A,

11, 12-dideoxy-3-O- decladinosyl-5-O-(2′-O-benzoyl-3′-N-desmethyl-3′-N-isopropyl)-6-O-methyl-3-oxo-12, 11-[oxycarbonyl-(3-(4-pyridin-3-yl)-imidazol-1-yl)-propyl)-imino)] -erythromycin A,

11, 12-dideoxy-3-O- decladinosyl-5-O-(3′-N-desmethyl-3′-N-isopropyl)-6-O-methyl-3-oxo-12, 11-[oxycarbonyl-(3-(4-pyridin-3-yl)-imidazol-1-yl)-propyl)-imino)] -erythromycin A,

11,12-dideoxy-3-O- decladinosyl-5-O-(3′-N-desmethyl-3′-N-2-fluoroethyl)-6-O-methyl-3-oxo-12,11-[oxycarbonyl-(4-(4-(4-amino-3,5-difluoro-phen-1-yl)-pyrazol-1-yl)-butyl)-imino)] -erythromycin A,

11, 12-dideoxy-3-O- decladinosyl-5-O-(3′-N-desmethyl-3′-N-ethyl)-6-O-methyl-3-oxo-12, 11-[oxycarbonyl-(3-(4-pyridin-3-yl)-imidazol-1-yl)-propyl)-imino)] -erythromycin A,

11, 12-dideoxy-3-O- decladinosyl-5-O-(2′-O-benzoyl-3′-N-desmethyl-3′-N-ethyl)-6-O-methyl-3-oxo-12, 11-[oxycarbonyl-(3-(4-pyridin-3-yl)-imidazol-1-yl)-propyl)-imino)] -erythromycin A,

11,12-dideoxy-3-O- decladinosyl-5-O-(3′-N-desmethyl-3′-N-isopropyl)-6-O-methyl-3-oxo-12,11-[oxycarbonyl-(5-(4-(2-amino-pyrimidin-5-yl)-pyrazol-1-yl)-pentyl)-imino)] -erythromycin A,

11, 12-dideoxy-3-O- decladinosyl-5-O-(2′-O-benzoyl -3′-N-desmethyl-3′-N-isopropyl)-6-O-methyl-3-oxo-12, 11-[oxycarbonyl-(4-(4-pyridin-3-yl)-imidazol-1-yl)-butyl)-imino)] -erythromycin A, or

11, 12-dideoxy-3-O- decladinosyl-5-O-(3′-N-desmethyl-3′-N-isopropyl)-6-O-methyl-3-oxo-12, 11-[oxycarbonyl-(4-(4-pyridin-3-yl)-imidazol-1-yl)-butyl)-imino)] -erythromycin A.

or pharmaceutically acceptable salts, pharmaceutically acceptable solvates, enantiomers, diastereomers or polymorphs thereof.

In yet another aspect, provided herein are pharmaceutical compositions comprising therapeutically effective amounts of one or more compounds of compounds described herein together with one or more pharmaceutically acceptable carriers, excipients, or diluents.

In another aspect, provided herein are methods for treating or preventing conditions caused by or contributed to by bacterial infections comprising administering to a mammal in need thereof therapeutically effective amounts of one or more compounds of compounds described herein.

The methods may include one or more of the following embodiments. For example, the condition can be selected from community acquired pneumonia, upper or lower respiratory tract infections, skin or soft tissue infections, acne vulgaris, hospital acquired lung infections, hospital acquired bone or joint infections, mastitis, catheter infection, foreign body, prosthesis infections or peptic ulcer disease. In another embodiment, the bacterial infection can be caused by gram positive, gram negative or anaerobic bacteria.

In yet another embodiment, the gram positive, gram negative or anaerobic bacteria can be selected from Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Propionibacterium acnes or Enterobactericeae. In a specific embodiment, the bacterium is cocci. In another specific embodiment, the cocci is drug resistant.

In another aspect, provided herein are methods for treating or preventing acne vulgaris and inflammatory conditions thereof comprising administering to a mammal in need thereof therapeutically effective amounts of one or more compounds of Formula I in combination with one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid, and other therapeutic agents, which can be used to treat acne or condition the skin.

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulfinyl, sulfonyl group or —NRₐ—, wherein Rₐ can be hydrogen, alkyl, alkenyl, alkynyl cycloalkyl or aryl. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further (referred herein as “substituted alkyl”) with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl (for R₆-R₉, alkyl is not substituted with aryl), heterocyclyl, heteroaryl, arylthio, thiol, alkylthio, aryloxy, nitro, aminosulfonyl, aminocarbonylamino, —NHC(═O)Rₖ, —NRₚR_{q}, —C(═O)NRₚR_{q}, —NHC(═O)NRₚR_{q}, —C(═O)heteroaryl, C(═O)heterocyclyl, —O—C(═O)NRₚR_{q} {wherein Rₚ and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, hydroxyamino, alkoxyamino or S(O)ₘR₆₆ (wherein m is an integer from 0-2 and R₆₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NRₚR_{q}, —C(═O)NRₚR_{q}, —OC(═O)NRₚR_{q}, —NHC(═O)NR_{fp}R_{q} (wherein Rₚ and R_{q} are the same as defined earlier), hydroxy, alkoxy, halogen, CF₃, cyano, and S(O)ₘR₆₆ (wherein m is an integer from 0-2 and R₆₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl,—C(═O)ORₚ (wherein Rₚ is the same as defined earlier), S(O)ₘR₆₆ (wherein m is an integer from 0-2 and R₆₆ is as defined earlier), or —C(═O)NRₚR_{q} (wherein Rₚ and R_{q} are as defined earlier)}. Unless otherwise constrained by the definition, all substituents may be substituted further by 1-3 substituents selected from alkyl, carboxy, carboxyalkyl, —NRₚR_{q}, —C(═O)NRₚR_{q}, —O —C(═O)NRₚR_{q} (wherein Rₚ and R_{q} are the same as defined earlier) hydroxy, alkoxy, halogen, CF₃, cyano, and S(O)ₘR₆₆ (wherein m is an integer from 0-2 and R₆₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. It can be optionally interrupted by atom(s) or group(s) independently chosen from oxygen, sulfur, phenylene, sulfinyl, sulfonyl and —NRₐ—, wherein Rₐ can be hydrogen, alkyl, alkenyl, alkynyl, cycloalkyl or aryl. In the event that alkenyl is attached to a heteroatom, the double bond cannot be alpha to the heteroatom. Alkenyl groups may be substituted further (referred to herein as “substituted alkenyl”) with one or more substituents selected from alkyl, alkenyl, alkynyl, alkoxy, cycloalkyl, cycloalkenyl, acyl, acylamino, acyloxy, —NHC(═O)Rₚ, —NRₚR_{q}, —C(═O)NRₚR_{q}, —NHC(═O)NRₚR_{q}, —O—C(═O)NRₚR_{q} (wherein Rₚ and R_{q} are the same as defined earlier), alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro, or SO₂R₆₆ (wherein R₆₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NRₚR_{q}, —C(═O)NRₚR_{q}, —O—C(═O)NRₚR_{q} (wherein Rₚ and R_{q} are the same as defined earlier) and —SO₂R₆₆ (where R₆₆ is same as defined earlier). Groups such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. It can be optionally interrupted by atom(s) or group(s) independently chosen from oxygen, sulfur, phenylene, sulfinyl, sulfonyl and —NRₐ—, where Rₐ can be hydrogen, alkyl, alkenyl, alkynyl, cycloalkyl or aryl. In the event that alkynyl is attached to a heteroatom, the triple bond cannot be alpha to the heteroatom. Alkynyl groups may be substituted further (referred to herein as “substituted alkynyl”) with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)Rₚ, —NRₚR_{q}, —NHC(═O)NRₚR_{q}, —C(═O)NRₚR_{q}, —C(═O)NRₚR_{q} (wherein Rₚ and R_{q} are the same as defined earlier), S(O)ₘR₆₆ (wherein m is an integer from 0-2 and R₆₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NRₚR_{q}, —C(═O)NRₚR_{q}, NHC(═O)NRₚR_{q}, —C(═O)NRₚR_{q} (wherein Rₚ and R_{q} are the same as defined earlier), cyano, or S(O)ₘR₆₆ (wherein m is an integer from 0-2 and R₆₆ is same as defined earlier). Groups such as ethynyl, (—C≡CH), propargyl (or propynyl, —CH₂C≡CH), and the like exemplify this term.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo [2.2.1] heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NRₚR_{q}, —NHC(═O)NRₚR_{q}, —NHC(═O)Rₚ, —C(═O)NRₚR_{q}, —O—C(═O)NRₚR_{q} (wherein Rₚ and R_{q} are the same as defined earlier), nitro, heterocyclyl, heteroaryl, heterocyclylalkyl, heteroarylalkyl, or S(O)ₘR₆₆ (wherein m is an integer from 0-2 and R₆₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NRₚR_{q}, —C(═O)NRₚR_{q}, —NHC(═O)NRₚR_{q}, —O—C(═O)NRₚR_{q} (wherein Rₚ and R_{q} are the same as defined earlier), cyano or S(O)ₘR₆₆ (wherein m is an integer from 0-2 and R₆₆ is same as defined earlier). As used herein the term “halogen or halo” refers to fluorine, chlorine, bromine or iodine.

As used herein the term “hydroxyl protected” includes, but is not limited to, acyl, aroyl, alkyl, aryl, butyldiphenylsilyl, methoxymethyl and methylthiomethyl, and the like.

As used herein the term “thio” refers to the group —SH.

The term “alkoxy” denotes the group O-alkyl or O-cycloalkyl, wherein alkyl and cycloalkyl are the same as defined above. Examples of alkoxy include, but are not limited to, methoxy, ethoxy, cyclopentoxy, and the like.

As used herein the term “thioalkyl” refers to —SR⁵ wherein R⁵ is alkyl or cycloalkyl.

As used herein the term “haloalkyl” refers to alkyl of which one or more hydrogen(s) is/are replaced by halogen.

The term “aryl” herein refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₛ (wherein Rₛ is hydrogen, alkyl, alkenyl, cycloalkyl, aralkyl, heterocyclylalkyl, heteroarylalkyl), NHC(═O)Rₚ, —NRₚR_{q}, —C(═O)NRₚR_{q}, —NHC(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined above. Examples of aralkyl include, but are not limited to, benzyl, napthylmethyl, phenethyl and phenylpropyl, and the like.

The terms “heterocycle” or “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, alkoxy, cycloalkyl, acyl, amino, optionally substituted aryl, alkoxy, alkaryl, cyano, nitro, oxo, carboxy, guanidine, haloalkyl, optionally substituted heterocyclyl, optionally substituted heterocyclylalkyl, heteroaryl, —CORₚ, —O—C(═O)Rₚ, —O—C(═O)ORₚ, —C(═O)NRₚR_{q}, S(O)ₘR₆₆, —O—C(═O)NRₚR_{q}, nitro, —NHC(═O)NRₚR_{q}, —NRₚR_{q} (wherein m, R₆₆, Rₚ and R_{q} are as defined earlier), —NHCORₚ, —NHSO₂Rₚ, and —SO₂NHRₚ, mercapto or thioalkyl.

Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocycles include, but not limited to, azabicyclohexyl, azetidinyl, benzoimidazolyl, 1,4-benzodioxanyl, 1,3-benzodioxolyl, benzoxazolyl, benzothiazolyl, benzothiazinyl, benzotriazolyl, benzoxazinyl, carbaxolyl, dihydrobenzofuryl, dihydroimidazolyl, dihydropyranyl, dihydrofuranyl, dihydroindolyl, dihydroisoxazolyl, dihydropyridinyl, dioxanyl, dioxolanyl, furyl, homopiperidinyl, imidazolyl, imidazolinyl, imidazolidinyl, imidazopyridinyl, indolinyl, indolyl, isoindole 1,3-dione, isoquinolinyl, isothiazolidinyl, isothiazolyl, isoxazolidinyl, isoxazolyl, morpholinyl, napthyridinyl, oxazolidinyl, oxazolyl, phenoxazinyl, phenothiazinyl, piperazinyl, piperidinyl, purinyl, pyrazinyl, pyrazolinyl, pyrazolyl, pyridinyl, pyridyl, pyrimidinyl, pyrrolidinyl, pyrrolinyl, pyrrolyl, pyrrolopyridinyl, quinolinyl, tetrahydrofuranyl, tetrahydropyranyl, tetrazolyl, thiazolidinyl and thiazolyl, and thienyl and the like.

As used herein the term “(heterocyclyl)alkyl” refers to heterocycle which is bonded to an alkylene chain, wherein heterocyclyl and alkyl are the same as defined above. Examples of heterocycle alkyl include, but are not limited to, isothiazolidinyl ethyl, isothiazolyl propyl, pyrazinyl methyl, pyrazolinyl propyl and pyridyl butyl, pyridyl methyl and the like.

As used herein the term “polymorphs” refers to all crystalline forms and amorphous forms of the compounds described herein. In addition, some of the compounds described herein may form solvates with water (i.e., hydrate, hemihydrate or sesquihydrate) or common organic solvents. Such solvates are also encompassed herein.

The phrase “pharmaceutically acceptable salts” denotes salts of the free base, which possess the desired pharmacological activity of the free base and which are neither biologically nor otherwise undesirable. Suitable pharmaceutically acceptable salts may be prepared from an inorganic or organic acid. Example of such inorganic acids include, but not limited to, hydrochloric, hydrobromic, hydroiodic, nitrous (nitrite salt), carbonic, sulfuric, phosphoric acid and like. Appropriate organic acids include, but not limited to, aliphatic, cycloaliphatic, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galacturonic acid and the like.

The term “pharmaceutically acceptable carriers” is intended to include non-toxic, inert solid, semi-solid or liquid filler, diluent, encapsulating material or formulation auxiliary of any type.

The compounds described herein include stereoisomers. The term “stereoisomer” refers to compounds, which have identical chemical composition, but differ with regard to arrangement of the atoms and the groups in space. These include enantiomers, diastereomers, geometrical isomers, atropisomer and comformational isomers. Geometric isomers may occur when a compound contains a double bond or some other feature that gives the molecule a certain amount of structural rigidity. An enantiomer is a stereoisomer of a reference molecule that is the nonsuperimposable mirror image of the reference molecule. A diastereomer is a stereoisomer of a reference molecule that has a shape that is not the mirror image of the reference molecule. An atropisomer is a conformation of a reference compound that converts to the reference compound only slowly on the NMR or laboratory time scale. Conformational isomers (or conformers or rotational isomers or rotamers) are stereoisomers produced by rotation about a bonds, and are often rapidly interconverting at room temperature. Racemic mixtures are also encompassed herein.

### DETAILED DESCRIPTION OF THE INVENTION

Compounds described herein may be prepared by techniques well known to one of ordinary skill in the art. In addition, compounds described herein may also be prepared by the following reaction sequences as depicted in Scheme I below.

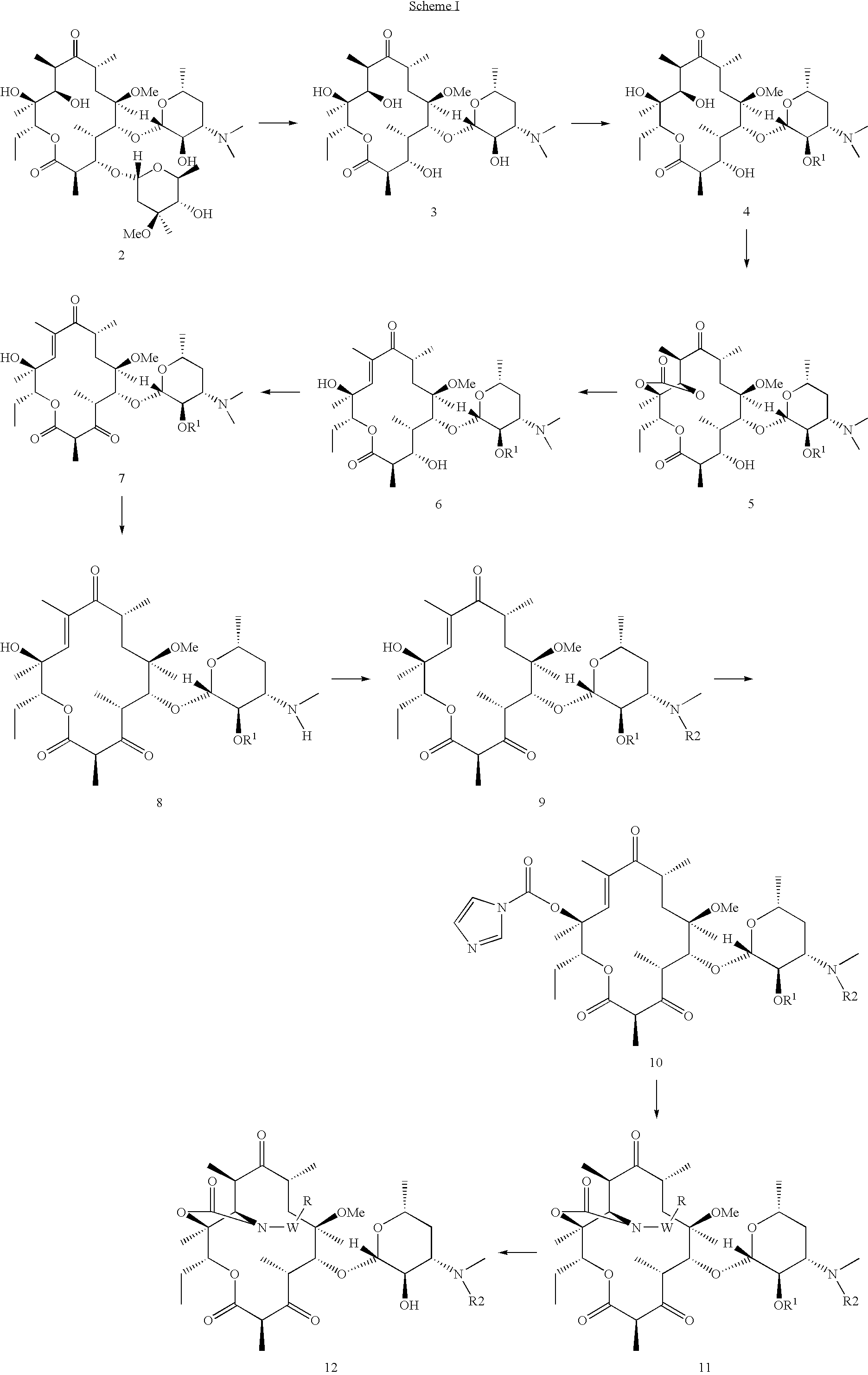

Compounds of Formula 12 can be prepared according to Scheme I. Thus, clarithromycin of Formula 2 can be hydrolyzed to form a compound of Formula 3. The compound of Formula 3 can be protected by reacting with one or more reagents of Formula R¹₂O or R¹X (wherein X is halogen) to form a compound of Formula 4 (wherein R¹═COPh). The compound of Formula 4 can be reacted with one or more reagents, for example, triphosgene, ethylene dicarbonate or a mixture thereof, to form a compound of Formula 5. The compound of Formula 5 can be reacted with one or more organic bases (for example, tetramethyl guanidine, trimethylamine or mixtures thereof) to form a compound of Formula 6. The compound of Formula 6 can be oxidized to form a compound of Formula 7. The compound of Formula 7 can be desmethylated at the 3′-N-dimethyl group to form a compound of Formula 8. The compound of Formula 8 can be alkylated by reacting with one or more reagents of Formula R²CHO, R²₂CO or R²X (wherein X is halogen) to form a compound of Formula 9 (wherein R² is the same as defined earlier). The compound of Formula 9 can be reacted with N,N′-carbonyldiimidazole to form a compound of Formula 10. The compound of Formula 10 can be reacted with a compound of Formula RWNH₂ to form a compound of Formula 11 (wherein W and R are the same as defined earlier). The compound of Formula 11 can be deprotected to form a compound of Formula 12.

Clarithromycin of Formula 2 can be hydrolyzed in the presence of an inorganic or organic acid, for example, hydrochloric acid, sulfuric acid or dichloroacetic acid.

The compound of Formula 3 can be hydroxyl protected by reacting with one or more reagents of Formula R¹₂O or R¹X in one or more solvents, for example, dichloromethane, dichloroethane, chloroform, carbon tetrachloride, ethyl acetate or mixtures thereof. The protection reactions can also be carried out in the presence of one or more organic bases, for example, triethylamine, diisopropylethylamine, pyridine, tributylamine, 4-(N-dimethylamino)pyridine or mixtures thereof.

The compound of Formula 4 can be reacted to form compounds of Formula 5 in one or more solvents, for example, chloroform, dichloromethane, carbon tetrachloride, dichloroethane or mixtures thereof. These reactions can also be carried out in the presence of one or more organic bases, for example, triethylamine, diisopropyl ethylamine, pyridine, tributylamine, 4-(N-dimethylamino)pyridine or mixtures thereof.

Compounds of Formula 5 can be reacted with one or more organic bases in one or more solvents, for example, dimethylformamide, tetrahydrofuran, dimethylsulfoxide or mixtures thereof.

Compounds of Formula 6 can be oxidized by reacting with one or more oxidizing agents, for example, Dess-Martin periodinane, N-chlorosuccinimide, pyridinium chlorochromate, Swern Oxidation reagent (oxalyl chloride and dimethylsulfoxide), Pfitzner-Moffatt Oxidation reagent (dicyclohexylcarbodiimide and dimethylsulfoxide), Jones Oxidation reagent (chromic acid, aqueous sulfuric acid and acetone), pyridinium dichromate, 1-ethyl-3-(3-dimethylaminopropyl)carbodiimide hydrochloride or mixtures thereof. N-Chlorosuccinamide can be used in combination with dimethyl sulfide and 1-ethyl-3(3-dimethylaminopropyl)carbodiimide hydrochloride can be used in combination with dimethylsulfoxide. Compounds of Formula 6 can also be oxidized in one or more solvents, for example, chloroform, dichloromethane, carbon tetrachloride, dimethylsulfoxide, dichloroethane or mixtures thereof.

Compounds of Formula 7 can be desmethylated in the presence of one or more desmethylating agents, for example, N-iodosuccinimide iodine in acetic acid, diisopropylazodicarboxylate or mixtures thereof. Such desmethylation reactions can also be carried out in one or more solvents, for example, acetonitrile, tetrahydrofuran, dichloromethane, dichloroethane, chloroform, carbon tetrachloride, ethyl acetate or mixtures thereof.

Desmethylation reactions can be quenched in the presence of one or more quenching agents, for example, sodium bisulfite, sodium carbonate, potassium carbonate, cesium carbonate, sodium acetate or mixtures thereof.

Compounds of Formula 8 can be alkylated with one or more reagents of Formula R²CHO, R²₂CO or R²X in one or more solvents, for example, dimethylformamide, acetonitrile, methanol, acetone, tetrahydrofuran or mixtures thereof. Alkylation reactions can also be carried out in the presence of one or more inorganic or organic bases, for example, sodium hydrogen carbonate, potassium carbonate, cesium carbonate, sodium hydride, pyridine, triethylamine, sodium acetate, sodium thiosulfate, diisopropyl ethylamine or mixtures thereof. Alkylation reactions can also be carried out in the presence of one or more reducing agents (for example, sodium cyanoborohydride, sodium borohydride, sodium triacetoxyborohydride or mixtures there) and in the presence of one or more organic acids (for example, acetic acid or dichloroacetic acid in a solvent, for example, methanol, ethanol, propanol, isopropanol or mixtures thereof).

Compounds of Formula 9 can be reacted with N,N′-carbonyldiimidazole in one or more solvents, for example, dimethylformamide, acetonitrile, tetrahydrofuran or mixtures thereof. This reaction can also be carried out in the presence of one or more inorganic bases, for example, sodium hydrogen carbonate, sodium acetate, sodium thiosulfate, potassium carbonate, cesium carbonate or sodium hydride.

Compounds of Formula 10 can be reacted with compounds of Formula RWNH₂ in one or more solvent systems, for example, acetonitrile/water, dimethylformamide/water, dimethylformamide or combinations thereof.

Compounds of Formula 11 can be deprotected in one or more alcohols, for example, methanol, ethanol, propanol, isopropanol or mixtures thereof.

In the above schemes, where specific reagents, for example, bases, acids oxidizing agents, solvents, etc., are described, it is to be understood that other reagents, e.g., bases, acids, oxidizing agents, solvents, etc., known to one of ordinary skill in the art may be used. Similarly, reaction temperatures and durations may be adjusted according to the desired needs without undue experimentation and well within the abilities of one of ordinary skill in the art. All the epimers, unless otherwise specified in the above schemes, are also encompassed herein.

Compounds described herein useful for such purpose are listed below:

5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyridin-3-yl-methyl-benzimidazol-1-yl)-butyl)-imino)] erythromycin A (Compound No. 1),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyridin-3-yl-imidazol-1-yl)-butyl)-imino)] erythromycin A (Compound No. 2),

5-O-(3′-N-desmethyl-3′-N-ethyl)desosaminyl-11,12-dideoxy-3-O-decladinosyl-6-O-methyl-3-oxo-12,11-[oxycarbonyl-((4-(imidazo[4,5-b]pyridin-1-yl)-butyl)-imino)] erythromycin A (Compound No. 3),

5-O-(3′-N-desmethyl-3′-N-ethyl)desosaminyl-11,12-dideoxy-3-O-decladinosyl-6-O-methyl-3-oxo-12,11-[oxycarbonyl-((4-(3,4-dihydro-2H-quinolin-1-yl)-butyl)-imino)] erythromycin A (Compound No. 4),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-tetrazol-1-yl-imidazol-1-yl)-butyl)-imino)] erythromycin A (Compound No. 5),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-3-yl-imidazol-1-yl)-butyl)-imino)] erythromycin A (Compound No. 6),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiophen-2-yl-imidazol-1-yl)-butyl)-imino)] erythromycin A (Compound No. 7),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-2-yl-imidazol-1-yl)-butyl)-imino)] erythromycin A (Compound No. 8),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyrazin-2-yl-thiazol-2-yl)-butyl)-imino)] erythromycin A (Compound No. 9),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyridin-3-yl-thiazol-2-yl)-butyl)-imino)] erythromycin A (Compound No. 10),

5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrazol-1-yl-pyridin-3-yl)-butyl)-imino)] erythromycin A (Compound No. 11),

5-O-(3′-N-desmethyl-3′-N-ethyl)desosaminyl-11,12-dideoxy-3-O-decladinosyl-6-O-methyl-3-oxo-12,11-[oxycarbonyl-((4-(6-imidazol-1-yl-pyridin-3-yl)-butyl)-imino)] erythromycin A (Compound No. 12),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-amino phenyl)-imidazol-1-yl)-butyl)-imino)] erythromycin A (Compound No. 13),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(6-pyrrol-1-yl)-pyridin-3-yl)-imidazol-1-yl)-butyl)-imino)] erythromycin A (Compound No. 14),

5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrrol-1-yl-pyridin-3-yl)-butyl)-imino)] erythromycin A (Compound No. 15),

5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyrrol-1-yl-thiazol-5-yl)-butyl)-imino)] erythromycin A(Compound No. 16),

5-O-(3′-N-desmethyl-3′-N-ethyl)desosaminyl-11,12-dideoxy-3-O-decladinosyl-6-O-methyl-3-oxo-12,11-[oxycarbonyl-((4-([1,4′]-biimidazol-1′-yl)-butyl)-imino)] erythromycin A (Compound No. 17),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiaophen-2-yl-pyridin-3-yl)-butyl)-imino)] erythromycin A (Compound No. 18),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)] erythromycin A (Compound No. 19),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)] erythromycin A(Compound No. 20),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dimethyl-amino)-phenyl)-imidazol-1-yl)-butyl)-imino)] erythromycin A (Compound No. 21),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-nicotinamido)-butyl)-imino)] erythromycin A(Compound No. 22),

5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-pyrrol-1-yl-thiazol-5-yl)-imidazol-1-yl)-butyl)-imino)] erythromycin A (Compound No. 23),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-benzamido)-butyl)-imino)] erythromycin A (Compound No. 24),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-acetyl-amino)-phenyl)-imidazol-1-yl)-butyl)-imino)] erythromycin A(Compound No. 25),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-isopropyl-amino)-phenyl)-imidazol-1-yl)-butyl)-imino)] erythromycin A(Compound No. 26),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-fluoro-phenyl)-imidazol-1-yl)-butyl)-imino)] erythromycin A (Compound No. 27),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-methoxy-phenyl)-imidazol-1-yl)-butyl)-imino)] erythromycin A (Compound No. 28),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(benzthiazol-2-yl)-benzamido)-butyl)-imino)] erythromycin A (Compound No. 29),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-isonicotinamido)-butyl)-imino)] erythromycin A (Compound No. 30),

5-O-(3-N-desmethyl-3′-N-ethyl)desosaminyl-11,12-dideoxy-3-O-decladinosyl-6-O-methyl-3-oxo-12,11-[oxycarbonyl-(4-(4-(N-(5-methyl-pyridin-2-yl)-benzamido)-butyl)-imino)] erythromycin A (Compound No. 31),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-imidazol-1-yl 1)-butyl)-imino)] erythromycin A (Compound No. 32),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-5-yl)-imidazol-1-yl 1)-butyl)-imino)] erythromycin A (Compound No. 33),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-4-yl)-imidazol-1-yl 1)-butyl)-imino)] erythromycin A (Compound No. 34),

5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-pyrazol-1-yl 1)-butyl)-imino)] erythromycin A (Compound No. 35),

11,12-dideoxy-3-O- decladinosyl-5-O-(3′-N-desmethyl-3′-N-2-fluoroethyl)-6-O-methyl-3-oxo-12,11-[oxycarbonyl-(4-(4-(3-amino-phen-1-yl)-imidazol-1-yl)-butyl)-imino)] -erythromycin A (Compound No.36),

11,12-dideoxy-3-O- decladinosyl-5-O-(3′-N-desmethyl-3′-N-2-fluoroethyl)-6-O-methyl-3-oxo-12,11-[oxycarbonyl-(4-(4-(2-amino-pyrimidin-5-yl)-imidazol-1-yl)-butyl)-imino)] -erythromycin A (Compound No. 37),

11,12-dideoxy-3-O- decladinosyl-5-O-(3′-N-desmethyl-3′-N-ethyl)-6-O-methyl-3-oxo-12,11-[oxycarbonyl-(4-(5-Nitro-indol-1-yl)-butyl)-imino] -erythromycin A (Compound No. 38),

11, 12-dideoxy-3-O- decladinosyl-5-O-(2′-O-benzoyl -3′-N-desmethyl-3′-N-isopropyl)-6-O-methyl-3-oxo-12, 11-[oxycarbonyl-(3-(4-pyridin-3-yl)-imidazol-1-yl)-propyl)-imino)] -erythromycin A (Compound No. 39),

11, 12-dideoxy-3-O- decladinosyl-5-O-(3′-N-desmethyl-3′-N-isopropyl)-6-O-methyl-3-oxo-12, 11- [oxycarbonyl-(3-(4-pyridin-3-yl)-imidazol-1-yl)-propyl)-imino)] -erythromycin A (Compound No. 40),

11,12-dideoxy-3-O- decladinosyl-5-O-(3′-N-desmethyl-3′-N-2-fluoroethyl)-6-O-methyl-3-oxo-12,11-[oxycarbonyl-(4-(4-(4-amino-3,5-difluoro-phen-1-yl)-pyrazol-1-yl)-butyl)-imino)] -erythromycin A (Compound No. 41),

11, 12-dideoxy-3-O- decladinosyl-5-O-(3′-N-desmethyl-3′-N-ethyl)-6-O-methyl-3-oxo-12, 11-[oxycarbonyl-(3-(4-pyridin-3-yl)-imidazol-1-yl)-propyl)-imino)] -erythromycin A (Compound No. 42),

11, 12-dideoxy-3-O- decladinosyl-5-O-(2′-O-benzoyl-3′-N-desmethyl-3′-N-ethyl)-6-O-methyl-3-oxo-12, 11-[oxycarbonyl-(3-(4-pyridin-3-yl)-imidazol-1-yl)-propyl)-imino)] -erythromycin A (Compound No. 43),

11,12-dideoxy-3-O- decladinosyl-5-O-(3′-N-desmethyl-3′-N-isopropyl)-6-O-methyl-3-oxo-12,11-[oxycarbonyl-(5-(4-(2-amino-pyrimidin-5-yl)-pyrazol-1-yl)-pentyl)-imino)] -erythromycin A (Compound No. 44),

11, 12-dideoxy-3-O- decladinosyl-5-O-(2′-O-benzoyl -3′-N-desmethyl-3′-N-isopropyl)-6-O-methyl-3-oxo-12, 11-[oxycarbonyl-(4-(4-pyridin-3-yl)-imidazol-1-yl)-butyl)-imino)] -erythromycin A (Compound No. 45),

11, 12-dideoxy-3-O- decladinosyl-5-O-(3′-N-desmethyl-3′-N-isopropyl)-6-O-methyl-3-oxo-12, 11-[oxycarbonyl-(4-(4-pyridin-3-yl)-imidazol-1-yl)-butyl)-imino)] -erythromycin A (Compound No. 46).

or pharmaceutically acceptable salts, pharmaceutically acceptable solvates, enantiomers, diastereomers or polymorphs thereof.

The compounds described herein can be pharmacologically active against gram-positive, gram-negative and anaerobic bacteria and accordingly, can be useful as antibacterial agents for treating bacterial infections in a patient in need thereof, for example, in a human or an animal. Because of their antibacterial activity, the compounds described herein may be administered to an animal for treatment by any route of administration, for example, orally, topically, rectally, internasally, or by parenteral route. Pharmaceutical compositions described herein comprise pharmaceutically effective amounts of compounds described herein formulated together with one or more pharmaceutically acceptable carriers, excipients or diluents.

Solid form preparations for oral administration include capsules, tablet, pills, powder, granules, cachets and suppositories. For solid form preparations, active compounds can be mixed with one or more inert, pharmaceutically acceptable excipients or carrier, for example, sodium citrate, dicalcium phosphate and/or fillers or extenders (for example, starches, lactose, sucrose, glucose, mannitol, silicic acid or mixtures thereof); binders, for example, carboxymethylcellulose, alginates, gelatins, polyvinylpyrrolidinone, sucrose, acacia or mixtures thereof; disintegrating agents, for example, agar-agar, calcium carbonate, potato starch, alginic acid, certain silicates, sodium carbonate or mixtures thereof; absorption acceletors, for example, quaternary ammonium compounds; wetting agents, for example, cetyl alcohol, glycerol mono stearate or mixtures thereof; adsorbants, for example, Kaolin; lubricants, for example, talc, calcium stearate, magnesium stearate, solid polyethyleneglycol, sodium lauryl sulfate or mixtures thereof; or mixtures thereof.

Capsules, tablets or pills may also comprise buffering agents.

Tablets, capsules, pills or granules can be prepared using one or more coatings or shells, for example, enteric coatings or other coatings known to one of ordinary skill in the art.

Liquid form preparations for oral administration include pharmaceutically acceptable emulsions, solutions, suspensions, syrups or elixirs. In such liquid form preparations, active compounds can be mixed with water or one or more other solvents, solubilizing agents or emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil), glycerol, fatty acid esters of sorbitan or mixtures thereof. Oral compositions can also include one or more adjuants, for example, wetting agents, emulsifying agents, suspending agents, sweetening agents, flavouring agents, perfuming agents or mixtures thereof.

Injectable preparations, for example, sterile injections, and aqueous suspensions may be formulated according to methods known to one of ordinary skill in the art, for example, using one or more suitable dispersing or wetting and suspending agents. Acceptable vehicles and solvents that may be employed include one or more of water, Ringer's solution, isotonic sodium chloride or mixtures thereof.

Dosage forms for tropical or transdermal administration of compounds described herein include ointments, pastes, creams, lotions, gels, powders, solutions, sprays, inhalants or patches. Such compounds can be admixed under sterile condition with one or more pharmaceutically acceptable carriers and optionally any preservatives or buffers as may be required. Ophthalmic formulations, eardrops, eye ointments, powders and solutions are also encompassed herein.

Pharmaceutical preparations may be in unit dosage form. In unit dosage form, preparations can be subdivided into unit doses containing appropriate quantities of active components. Unit dosage forms can be packaged preparations containing discrete capsules, powders, in vials or ampoules, ointments, capsules, sachets, tablets, gels, creams or any combination and number of such packaged forms.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Example 1

### Preparation of a Compound of Formula 3

Clarithromycin (25 g, 33.4 mmol) was added portionwise to an aqueous solution of hydrochloric acid at ambient temperature. The reaction mixture was neutralized with solid sodium bicarbonate and the aqueous layer was extracted with ethyl acetate. The organic layer was washed with water followed by brine, and dried over anhydrous sodium sulphate and the solvent was removed under reduced pressure to afford crude product. The crude product was crystallized from ethyl acetate and hexane mixture to yield the title compound.

### Example 2

### Preparation of a Compound of Formula 4

Benzoic anhydride (2.5 equiv.) followed by triethylamine (6 equiv.) was added to a solution of a compound of Formula 2 (1 equiv.) in dichloromethane and stirred at ambient temperature for about 40 hours. The reaction was quenched by addition of sodium bicarbonate solution. The aqueous layer was extracted with dichloromethane, washed successively with water followed by brine, and dried over anhydrous sodium sulphate and the solvent was removed under reduced pressure to form a crude product. The crude product was crystallized from ethyl acetate and hexane mixture to form the title compound.

### Example 3

### Preparation of a Compound of Formula 5

Triphosgene (1.5 equiv.) was added to a solution of a compound of Formula 3 (1 equiv.) in dichloromethane and pyridine (15 equiv.) was slowly added. The reaction mixture was stirred for about 4 hours and then quenched by addition of ice-cold water. The reaction mixture was diluted with dichloromethane, washed with water followed by brine, dried over anhydrous sodium sulphate and concentrated under reduced pressure to yield the title compound.

### Example 4

### Preparation of a Compound of Formula 6

Tetramethyl guanidine (2.2 equiv.) was added to a solution a compound of Formula 4 (1 equiv.) in dimethylformamide and the mixture was heated to about 70° C. and stirred for about 10 hours. The reaction mixture was cooled to ambient temperature. The organic layer was extracted with ethyl acetate, washed with water followed by brine, dried over anhydrous sodium sulphate and concentrated under reduced pressure to yield the title compound.

### Example 5

### Preparation of a Compound of Formula 7

Dess-Martine Periodinane (2.5 equiv.) was added to a solution a compound of Formula 5 (1 equiv.) in dichloromethane and refluxed for about one hour. The reaction was cooled to ambient temperature and quenched by addition of saturated aqueous potassium carbonate solution followed by saturated sodium thiosulphate solution and stirred. The aqueous layer was separated and extracted with dichloromethane. The dichloromethane layer was washed with water followed by brine, dried over anhydrous sodium sulphate and concentrated under reduced pressure to yield the title compound.

### Example 6

### Preparation of a Compound of Formula 8

N-iodosuccinimide (2 equiv.) was added to a solution a compound of Formula 6 (1 equiv.) in dry acetonitrile:dichloromethane (2:1) at about 0° C. and the reaction mixture was brought to ambient temperature with stirring. Sodium bisulphite solution was added followed sodium carbonate solution with stirring. Dichloromethane was evaporated under reduced pressure. The aqueous layer was extracted with ethyl acetate, washed successively with water followed by brine, and dried over anhydrous sodium sulphate. The solvent was removed under reduced pressure to yield the title compound.

### Example 7

### Preparation of a Compound of Formula 9

Solid sodium hydrogen carbonate (5 equiv.) and ethyl iodide (6 equiv.) were added to a solution a compound of Formula 8 (1 equiv.) in acetonitrile under argon at ambient temperature and the mixture was stirred for about 24 hours. The reaction was quenched by the addition of water, diluted with ethyl acetate and washed with water followed by brine, dried over anhydrous sodium sulphate and concentrated under reduced pressure to yield a crude product. The crude product was purified by silica gel column chromatography (thoroughly neutralized with triethylamine) using 10-15% acetone in hexane to yield the title compound.

Alternatively, when R² is isopropyl, the following procedure can be used to form a compound of Formula 9. In particular, acetone (5 equiv.) and acetic acid were added (2 equiv.) to a solution a compound of Formula 8 (1 equiv.) in methanol and stirred at ambient temperature for 1 hour. Sodium cyanoborohydride (5 equiv.) was added portionwise and the reaction mixture was stirred at ambient temperature for 15 hours. The excess solvent was evaporated to dryness and the resulting compound was dissolved to methylene chloride and washed with water followed by brine. The organic layer was dried over anhydrous sodium sulphate, filtered and concentrated to form a solid crude product. The crude product was purified by silica gel column chromatography (thoroughly neutralized with triethylamine) using 10-15% acetone in hexane to yield the title compound.

### Example 8

### Preparation of a Compound of Formula 10

N, N′-carbonyldiimidazole (3 equiv.) was added to a solution a compound of Formula 8 (1 equiv.) in dimethylformamide:tetrahydrofuran (3:2) at ambient temperature, cooled, and sodium hydride (3 equiv.) was added in portions and stirred. The reaction mixture was quenched by adding water and then extracted with ethyl acetate. The organic layer was washed with water followed by brine, dried over anhydrous sodium sulphate and concentrated under reduced pressure to yield the title compound.

### Example 9

### Preparation of a Compound of Formula 11

The compound of Formula 9 (1 equiv.) and R—W—NH₂ (2 equiv.) were taken in water in acetonitrile and heated at 70° C. and stirred for about 20 hours. The reaction mixture was cooled to attain ambient temperature and acetonitrile was removed under reduced pressure. The resulting residue was taken in ethyl acetate and washed with water followed by brine, dried over anhydrous sodium sulphate, and filtered. The filtrate was collected and concentrated under reduced pressure. The compound thus obtained was purified by silica gel column chromatography (thoroughly neutralized with triethylamine) using 25-30% acetone in hexane to yield the title compound.

### Example 10

### Preparation of a Compound of Formula 12

The compound of Formula 10 was taken in methanol and refluxed. The reaction mixture was cooled to attain ambient temperature and methanol was evaporated under reduced pressure. The compound thus obtained was purified by silica gel column using 2-10% methanol in dichloromethane to yield the title compound.

The following compounds were prepared following the procedures described above:

Compound No. 1: 5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyridin-3-yl-methyl-benzimidazol-1-yl)-butyl)-imino)] erythromycin A; Mass (M+1): 890.54; M.P.: 170-173 (in ° C.)

Compound No. 2: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyridin-3-yl-imidazol-1-yl)-butyl)-imino)] erythromycin A; Mass (M+1): 826.6; M.P.: 61-62 (in ° C.)

Compound No. 3: 5-O-(3′-N-desmethyl-3′-N-ethyl)desosaminyl-11,12-dideoxy-3-O-decladinosyl-6-O-methyl-3-oxo-12,11-[oxycarbonyl-((4-(imidazo[4,5-b]pyridin-1-yl)-butyl)-imino)] erythromycin A; Mass (M+1): 800; M.P.: 108-110 (in ° C.)

Compound No. 4: 5-O-(3′-N-desmethyl-3′-N-ethyl)desosaminyl-11,12-dideoxy-3-O-decladinosyl-6-O-methyl-3-oxo-12,11-[oxycarbonyl-((4-(3,4-dihydro-2H-quinolin-1-yl)-butyl)-imino)] erythromycin A; Mass (M+1): 814.79; M.P.: 75-77 (in ° C.)

Compound No. 5: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-tetrazol-1-yl-imidazol-1-yl)-butyl)-imino)] erythromycin A; Mass (M+1): 817.4; M.P.: 109-111 (in ° C.)

Compound No. 6: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-3-yl-imidazol-1-yl)-butyl)-imino)] erythromycin A; Mass (M+1): 815.4; M.P.: 105-107 (in ° C.)

Compound No. 7: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiophen-2-yl-imidazol-1-yl)-butyl)-imino)] erythromycin A; Mass (M+1): 831.4; M.P.: 113-115 (in ° C.)

Compound No. 8: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-2-yl-imidazol-1-yl)-butyl)-imino)] erythromycin A; Mass (M+1): 815.4; M.P.: 101-103 (in ° C.)

Compound No. 9: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyrazin-2-yl-thiazol-2-yl)-butyl)-imino)] erythromycin A; Mass (M+1): 844.4; M.P.: 70-71 (in ° C.)

Compound No. 10: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyridin-3-yl-thiazol-2-yl)-butyl)-imino)] erythromycin A; Mass (M+1): 843.4; M.P.: 86-87 (in ° C.)

Compound No. 11: 5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrazol-1-yl-pyridin-3-yl)-butyl)-imino)] erythromycin A; Mass (M+1): 826.4; M.P.: 93-94 (in ° C.)

Compound No. 12: 5-O-(3′-N-desmethyl-3′-N-ethyl)desosaminyl-11,12-dideoxy-3-O-decladinosyl-6-O-methyl-3-oxo-12,11-[oxycarbonyl-((4-(6-imidazol-1-yl-pyridin-3-yl)-butyl)-imino)] erythromycin A; Mass (M+1): 826.4; M.P.: 98-99 (in ° C.)

Compound No. 13: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-amino phenyl)-imidazol-1-yl)-butyl)-imino)] erythromycin A; Mass (M+1): 840.4; M.P.: 89-91 (in ° C.)

Compound No. 14: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-(6-pyrrol-1-yl)-pyridin-3-yl)-imidazol-1-yl)-butyl)-imino)] erythromycin A; Mass (M+1): 891.5; M.P.: 91-92 (in ° C.)

Compound No. 15: 5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrrol-1-yl-pyridin-3-yl)-butyl)-imino)] erythromycin A; Mass (M+1): 825.4; M.P.: 95-96 (in ° C.)

Compound No. 16: 5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyrrol-1-yl-thiazol-5-yl)-butyl)-imino)] erythromycin A; Mass (M+1): 831.3; M.P.: 87-88 (in ° C.)

Compound No. 17: 5-O-(3′-N-desmethyl-3′-N-ethyl)desosaminyl-11,12-dideoxy-3-O-decladinosyl-6-O-methyl-3-oxo-12,11-[oxycarbonyl-((4-([1,4′]-biimidazol-1′-yl)-butyl)-imino)] erythromycin A; Mass (M+1): 815.5; M.P.: 79-80 (in ° C.)

Compound No. 18: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiaophen-2-yl-pyridin-3-yl)-butyl)-imino)] erythromycin A, Mass (M+1): 842.3; M.P.: 89-91 (in ° C.)

Compound No. 19: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)] erythromycin A; Mass (M+1): 854.5; M.P.: 103-105 (in °C.)

Compound No. 20: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)] erythromycin A; Mass (M+1): 920.5; M.P.: 96-98 (in ° C.)

Compound No. 21: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dimethyl-amino)-phenyl)-imidazol-1-yl)-butyl)-imino)] erythromycin A; Mass (M+1): 868.5; M.P.: 96-97.5 (in ° C.)

Compound No. 22: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-nicotinamido)-butyl)-imino)] erythromycin A; Mass (M+1): 886.3; M.P.: 114-115 (in ° C.)

Compound No. 23: 5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-pyrrol-1-yl-thiazol-5-yl)-imidazol-1-yl)-butyl)-imino)] erythromycin A; Mass (M+1): 897.4; M.P.: 117-118 (in ° C.)

Compound No. 24: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-benzamido)-butyl)-imino)] erythromycin A; Mass (M+1): 885.4; M.P.: 103-105 (in ° C.)

Compound No. 25: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-acetyl-amino)-phenyl)-imidazol-1-yl)-butyl)-imino)] erythromycin A; Mass (M+1): 882.5; M.P.: 94-96 (in ° C.)

Compound No. 26: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-isopropyl-amino)-phenyl)-imidazol-1-yl)-butyl)-imino)] erythromycin A; Mass (M+1): 882.5; M.P.: 86-88 (in ° C.)

Compound No. 27: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-fluoro-phenyl)-imidazol-1-yl)-butyl)-imino)] erythromycin A; Mass (M+1): 843.4; M.P.: 92-95 (in ° C.)

Compound No.28: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-methoxy-phenyl)-imidazol-1-yl)-butyl)-imino)] erythromycin A; Mass (M+1): 855.4; M.P.: 100-102 (in ° C.)

Compound No. 29: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(benzthiazol-2-yl)-benzamido)-butyl)-imino)] erythromycin A; Mass (M+1): 935.5; M.P.: 128-129 (in ° C.)

Compound No. 30: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-isonicotinamido)-butyl)-imino)] erythromycin A; Mass (M+1): 886.4; M.P.: 102-104 (in ° C.)

Compound No. 31: 5-O-(3′-N-desmethyl-3′-N-ethyl)desosaminyl-11,12-dideoxy-3-O-decladinosyl-6-O-methyl-3-oxo-12,11 -[oxycarbonyl-(4-(4-(N-(5-methyl-pyridin-2-yl)-benzamido)-butyl)-imino)] erythromycin A; Mass (M+1): 893.5; M.P.: 110-112 (in ° C.)

Compound No. 32: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-imidazol-1-yl 1)-butyl)-imino)] erythromycin A; Mass (M+1): 842.4; M.P.: 124-125 (in ° C.)

Compound No. 33: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-5-yl)-imidazol-1-yl 1)-butyl)-imino)] erythromycin A; Mass (M+1): 841.4; M.P.: 97-99 (in ° C.)

Compound No. 34: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-4-yl)-imidazol-1-yl 1)-butyl)-imino)] erythromycin A; Mass (M+1): 841.4; M.P.: 124-125 (in ° C.)

Compound No. 35: 5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-pyrazol-1-yl 1)-butyl)-imino)] erythromycin A; Mass (M+1): 842.6; M.P.: 108-110 (in ° C.)

Compound No. 36: 11,12-dideoxy-3-O- decladinosyl-5-O-(3′-N-desmethyl-3′-N-2-fluoroethyl)-6-O-methyl-3-oxo-12,11-[oxycarbonyl-(4-(4-(3-amino-phen-1-yl)-imidazol-1-yl)-butyl)-imino)] -erythromycin A; Mass (M+1): 858.12;

Compound No. 37: 11,12-dideoxy-3-O- decladinosyl-5-O-(3′-N-desmethyl-3′-N-2-fluoroethyl)-6-O-methyl-3-oxo-12,11-[oxycarbonyl-(4-(4-(2-amino-pyrimidin-5-yl)-imidazol-1-yl)-butyl)-imino)] -erythromycin A ;Mass (M+1): 860.00;

Compound No. 38: 11,12-dideoxy-3-O- decladinosyl-5-O-(3′-N-desmethyl-3′-N-ethyl)-6-O-methyl-3-oxo-12,11-[oxycarbonyl-(4-(5-Nitro-indol-1-yl)-butyl)-imino] -erythromycin A; Mass (M+1): 841.4; M.P.: 97-99 (in ° C.) Mass (M+1): 845.00;

Compound No. 39: 11, 12-dideoxy-3-O- decladinosyl-5-O-(2′-O-benzoyl -3′-N-desmethyl-3′-N-isopropyl)-6-O-methyl-3-oxo-12, 11-[oxycarbonyl-(3-(4-pyridin-3-yl)-imidazol-1-yl)-propyl)-imino)] -erythromycin A; Mass (M+1): 930.86;

Compound No. 40: 11, 12-dideoxy-3-O- decladinosyl-5-O- (3′-N-desmethyl-3′-N-isopropyl)-6-O-methyl-3-oxo-12, 11-[oxycarbonyl-(3-(4-pyridin-3-yl)-imidazol-1-yl)-propyl)-imino)] -erythromycin A; Mass (M+1): 826.80;

Compound No. 41: 11,12-dideoxy-3-O- decladinosyl-5-O-(3′-N-desmethyl-3′-N-2-fluoroethyl)-6-O-methyl-3-oxo-12,11-[oxycarbonyl-(4-(4-(4-amino-3,5-difluoro-phen-1-yl)-pyrazol-1-yl)-butyl)-imino)] -erythromycin A; Mass (M+1): 876.80;

Compound No. 42: 11, 12-dideoxy-3-O- decladinosyl-5-O- (3′-N-desmethyl-3′-N-ethyl)-6-O-methyl-3-oxo-12, 11-[oxycarbonyl-(3-(4-pyridin-3-yl)-imidazol-1-yl)-propyl)-imino)] -erythromycin A; Mass (M+1): 812.7;

Compound No. 43: 11, 12-dideoxy-3-O- decladinosyl-5-O- (2′-O-benzoyl 3′-N-desmethyl-3′-N-ethyl)-6-O-methyl-3-oxo-12, 11-[oxycarbonyl-(3-(4-pyridin-3-yl)-imidazol-1-yl)-propyl)-imino)] -erythromycin A ;Mass (M+1): 916.9;

Compound No. 44: 11,12-dideoxy-3-O- decladinosyl-5-O-(3′-N-desmethyl-3′-N-isopropyl)-6-O-methyl-3-oxo-12,11-[oxycarbonyl-(5-(4-(2-amino-pyrimidin-5-yl)-pyrazol-1-yl)-pentyl)-imino)] -erythromycin A; Mass (M+1): 870.85;

Compound No.45: 11, 12-dideoxy-3-O- decladinosyl-5-O- (2′-O-benzoyl -3′-N-desmethyl-3′-N-isopropyl)-6-O-methyl-3-oxo-12, 11-[oxycarbonyl-(4-(4-pyridin-3-yl)-imidazol-1-yl)-butyl)-imino)] -erythromycin A; Mass (M+1): 944.98;

Compound No.46: 11, 12-dideoxy-3-O- decladinosyl-5-O- (3′-N-desmethyl-3′-N-isopropyl)-6-O-methyl-3-oxo-12, 11-[oxycarbonyl-(4-(4-pyridin-3-yl)-imidazol-1-yl)-butyl)-imino)] -erythromycin A; Mass (M+1): 840.73;

### Example 11

### Pharmacological Activity

Compounds described herein displayed antibacterial activity in vitro particularly against strains that are resistant to macrolides either due to efflux (mef strains) or ribosomal modification (erm) strains. These compounds are useful in treating community acquired pneumonia, upper and lower respiratory tract infections, skin and soft tissue infections, hospital acquired lung infections, bone and joint infections, and other bacterial infections, for example, mastitis, catheter infection, foreign body, prosthesis infections or peptic ulcer disease.

Minimum inhibitory concentration (MIC) has been an indicator of in vitro antibacterial activity widely used in the art.

### Procedure:

### Medium

a) Cation adjusted Mueller Hinton Agar (MHA-Difco)
b) Trypticase Soya Agar (TSA)
Inoculum preparation

Cultures were streaked on TSA for aerobic cultures and MHA with 5% sheep blood for fastidious cultures. Aerobic cultures were incubated at 37° C. for about 18-24 hours. Fastidious cultures were incubated CO₂ incubation (5% CO₂) at 37° C. for about 18-24 hours. Three to four well-isolated colonies were taken and saline suspensions were prepared in sterile densimat tubes. The turbidity of the culture was adjusted to 0.5-0.7 Mc Farland standard (1.5×10⁸ CFU/mL). The cultures were diluted 10 fold in saline to obtain inoculum sizes of approximately 1-2×10⁷ organisms/mL.

Preparation of drug concentration

1 mg/mL concentration of stock solution of drugs was prepared in dimethylsulfoxide/distilled water/solvent given in National Committee for Clinical Laboratory Standards (NCCLS) manual. Serial two-fold dilutions of the compounds and standard drugs were prepared as per NCCLS manual.

The stock solution was changed according to the need of the experiment.

### Preparation of Agar Plates

Two mL of respective drug concentration was added to 18 mL of Molten Mueller Hinton agar to achieve the required range, for example 0.015 μg/mL -16 μg/mL. For fastidious cultures 1 mL of sheep blood was added in Molten Mueller Hinton agar.

MHA and MHA with 5% sheep blood plates without antibiotic for each set were prepared for controls. One MHA and MHA with 5% sheep blood plate without antibiotic for determining quality check for media was prepared.

### Preparation of Teflon Template

1 μL of each culture on each plate was replicated with the help of a replicator (i.e., Denley's multipoint replicator). The spots were allowed to dry and the plates were incubated for about 18-24 hours at 37° C. Fastidious cultures were incubated at 37° C. in a CO₂ incubator. The results were noted comparing with the control plates.

### Endpoint Definition

The concentration of drug at which there was complete disappearance of growth spot or formation of less than 10 colonies per spot was considered as Minimum Inhibitory Concentration (MIC).

The MICs of Quality Control (QC) strains were plotted on the QC chart for agar dilution method. If the MICs were within the range, the results interpreted by comparing MICs of standards against all organisms with those of test compounds.

### Precautions & Quality Control Measures

Quality Control Strains Staphylococcus aureus ATCC 29213
Enterococcus faecalis ATCC 29212
Eschericia coli ATCC 25922
Pseudomonas aeruginosa ATCC 27853
All 60 cultures were visually checked for purity.

Media Control: NCCLS disc diffusion assay using 10 μg discs of Gentamicin (Difco) against Pseudomonas aeruginosa ATCC 27853. A zone diameter of 16-21 mm was considered for optimum cation (Magnesium and Calcium) content of the media. The diameter was plotted in the media QC chart.

### References:

National Committee for Clinical Laboratory Standards (NCCLS), Methods for Dilution Antimicrobial Susceptibility Tests for Bacteria That Grow Aerobically—Fifth Edition; Approved Standard. M7-A5, Vol.20. No. 2 (January 2000).

National Committee for Clinical Laboratory Standards, Performance Standards for Antimicrobial Susceptibility Testing—Twelfth informational supplement, M 100-12, Vol. 22 No. 1 (January 2002).

Results: Certain compounds described herein were found to have activity against one or more microbial strains, for example, Staphylococcus aureus, Streptococcus pneumoniae, Haemophilus influenzae, Moraxella catarrhalis, Streptococcus pyogenes, enterococci species, Helicobacter pylori, E. faecalis or combination thereof.

Certain compounds exhibited MIC against S. pneumoniae of about 0.008 μg/mL to about 16 μg/mL, from about 0.008 μg/mL to about 1 μg/mL, from about 0.008 μg/mL to about 0.125 μg/mL, and even from about 0.008 μg/mL to about 0.03 μg/mL.

Certain compounds exhibited MIC against H. influenzae of about 0.03 μg/mL to about 16 μg/mL, from about 0.03 μg/mL to about 2 μg/mL, from about 0.03 μg/mL to about 0.25 μg/mL, and even from about 0.03 μg/mL to about 0.125 μg/mL.

Certain compounds exhibited MIC against Moraxella of about 0.03 μg/mL to about 4 μg/mL, from about 0.03 μg/mL to about 1 μ/mL, from about 0.03 μg/mL to about 0.25 μg/mL, and even from about 0.03 μg/mL to about 0.125 μg/mL.

Certain compounds exhibited MIC against S. pyogenes of about 0.008 μg/mL to about 16 μg/mL, from about 0.008 μg/mL to about 1 μg/mL, from about 0.008 μg/mL to about 0.125 μg/mL, and even from about 0.008 μg/mL to about 0.03 μg/mL.

### Example 12

### Pharmacological Activity (Acne Vulgaris)

Method: Minimum inhibitory concentration (MIC) a compound described herein and standard drugs are determined against clinical and ATCC isolates (n=6) of Propionibacterium acnes[Propionibacterium acnes I (Sensitive), Propionibacterium acnes II (Sensitive), Propionibacterium acnes 6523, Propionibacterium acnes ATCC 6919 and two isolates of Propionibacterium acnes (Resistant)] as per NCCLS guidelines (M 11-A5) by agar dilution method. Brucella agar (Difco) with hemin (5 μg/ml) and vitamin K1 (1 μg/ml) supplemented with 5% (v/v) laked sheep blood is used as media. Stock concentrations of standard drug are prepared in respective diluents as per NCCLS guidelines. Stock solution (1 mg/ml) of NCEs is prepared in DMSO and serial two fold dilutions of drug are prepared and mixed with agar. Inoculum is prepared from freshly grown isolates on brucella blood agar and their turbidity is adjusted to approximately 0.5 McFarland. Replicate 1 μL of cultures on agar plate with the help of a replicator (Denley's multipoint replicator). All plates are incubated at 37° C. for 48 hours in anaerobic jar under anaerobic condition created by using Anaxomat.

We claim:

1. A compound having the structure of Formula I, 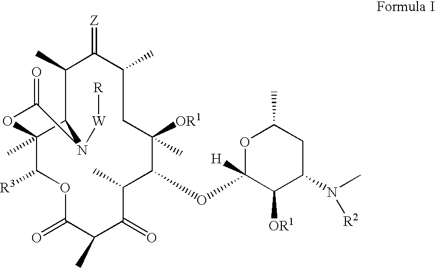
or a pharmaceutically acceptable salt, pharmaceutically acceptable solvate, enantiomer, diastereomer or polymorph thereof, wherein:
R¹ is hydrogen or a hydroxyl-protecting group;
R² is C₂-C₆ alkyl, alkenyl, alkynyl, cycloalkyl, aryl, heterocycle, aralkyl, (heterocyclic)alkyl or COR⁴, wherein
R⁴ is hydrogen, alkyl or aralkyl;
W is (CH₂)_{q}, CR⁵R⁶, NR⁵ or S)₂, wherein
q is an integer of from 2 to 10;
R⁵ and R⁶ are independently hydrogen or alkyl;
(CH₂)_{q} is optionally interrupted by one or more of unsaturated bonds, oxygen, sulfur, CO, CS, SO₂, NR₁ or combination thereof;
one or more hydrogen atoms of (CH₂)_{q} group is optionally replaced by halogen, alkyl, hydroxyl or alkoxy, wherein
R₁ is hydrogen, alkyl, alkenyl, alkynyl, COR₂ or (CH₂)ₘR₂;
R₂ is alkyl, alkenyl, alkynyl, aryl or heterocycle; and
m is an integer of from 2 to 4;
R is R₆NCOR₇, aryl or heterocycles, wherein
R₆ and R₇ are independently aryl or heterocycle;
R³ is alkyl, alkenyl or alkynyl;
R′ is alkyl or —(CH₂)ᵣ—U, wherein
r is an integer of from 1 to 4; and
U is alkenyl or alkynyl; and
Z is oxygen, sulfur or NOR⁴, wherein
R⁴ is the same as defined earlier.
The compound of claim 1, wherein:
R¹ is hydrogen;
R² and R³ is ethyl;
W is (CH₂)_{q}, wherein (CH₂)_{q} is the same as defined earlier;
R′ is methyl;
Z is oxygen; and
R is R₆NCOR₇, 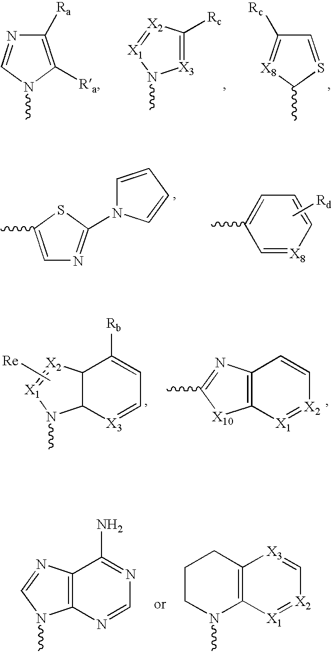
wherein X₁ to X₃ are independently CH or N;.
X₄ to X₈ are independently CH, CR₃ or N;
X₉ is O, S, N, NH or CH;
X₁₀ is NH or S;
Rₐ is optionally substituted thienyl, furyl, pyrazolyl, oxazolyl, tetrazolyl, imidazolyl, pyridinyl, fluoropyridinyl, chloropyridinyl, pyridinyl, pyrazinyl, pyrimidinyl, pyrimidinyl, quinolinyl, pyrrolo-pyridyl, pyrrolo-thiazolyl or phenyl wherein optional substituent(s) can be methyl, halogen, methoxy, NR₄R₅ [wherein R₄ and R₅ are independently hydrogen, methyl, isopropyl, cyclopropyl, acetyl];
R′ₐ is hydrogen or furyl;
R_{b} is hydrogen or NR₄R₅ [wherein R₄ and R₅ are independently hydrogen, methyl, isopropyl, cyclopropyl, acetyl];
R_{c} is hydrogen, optionally substituted thienyl, furyl, pyrazolyl, pyrazinyl, pyridinyl, pyridinyl, pyrimidinyl, pyrrolyl, imidazolyl or phenyl wherein optional substituent(s) can be methyl, halogen, methoxy, NR₄R₅ [wherein R₄ and R₅ can be independently hydrogen, methyl, isopropyl, cyclopropyl, acetyl];
R_{d} is thienyl, pyrazolyl, imidazolyl, triazolyl, pyrrolyl or tetrahydrofuryl;
Rₑ is (heterocyclic)alkyl; and
R₆ and R₇ are independently aryl or heterocyclyl.

3. A compound selected from:
5-O-(3′-N-desmethyl-3′-N-ethyl)desosaminyl-11,12-dideoxy-3-O-decladinosyl-6-O-methyl-3-oxo-12,11-[oxycarbonyl-((4- (2-pyridin-3-yl-methyl-benzimidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyridin-3-yl-methyl-benzimidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyridin-3-yl-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(imidazo[4,5-b]pyridin-1-yl)-butyl)-imino)] erythromycin A, 10856
5-O-(3′-N-desmethyl-3′-N-ethyl)desosaminyl-11,12-dideoxy-3-O-decladinosyl-6-O-methyl-3-oxo-12,11-[oxycarbonyl-((4-(3,4-dihydro-2H-quinolin-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-tetrazol-1-yl-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-3-yl-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiophen-2-yl-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-furan-2-yl-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyrazin-2-yl-thiazol-2-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-pyridin-3-yl-thiazol-2-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrazol-1-yl-pyridin-3-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(6-imidazol-1-yl-pyridin-3-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-amino phenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-(6-pyrrol-1-yl)-pyridin-3-yl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(6-pyrrol-1-yl-pyridin-3-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(2-pyrrol-1-yl-thiazol-5-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-([1,4′]-biimidazol-1′-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-thiaophen-2-yl-pyridin-3-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-(4-methyl-3-aminophenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dicyclopropyl-amino)-phenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-(3-(N,N-dimethyl-amino)-phenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-nicotinamido)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-((4-(4-(2-pyrrol-1-yl-thiazol-5-yl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-benzamido)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-acetyl-amino)-phenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(3-(N-isopropyl-amino)-phenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-fluoro-phenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(4-methoxy-phenyl)-imidazol-1-yl)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(benzthiazol-2-yl)-benzamido)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(thiazol-2-yl)-isonicotinamido)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(N-(5-methyl-pyridin-2-yl)-benzamido)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-imidazol-1-yl 1)-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-5-yl)-imidazol-1-yl 1-butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyridin)-4-yl)-imidazol-1-yl 1)butyl)-imino)] erythromycin A,
5-O-(3′-N-desmethyl-3′-N-ethyl)desosaminyl-11,12-dideoxy-3-O-decladinosyl-6-O-methyl-3-oxo-12,11-[oxycarbonyl-(4-(4-(2-amino-pyrimidin)-5-yl)-pyrazol-1-yl 1)-butyl)-imino)] erythromycin A,
11,12-dideoxy-3-O-decladinosyl-5-O-(3′-N-desmethyl-3′-N-2-fluoroethyl)-6-O-methyl-3-oxo-12,11-[oxycarbonyl-(4-(4-(3-amino-phen-1-yl)-imidazol-1-yl)-butyl)-imino)] -erythromycin A,
11,12-dideoxy-3-O-decladinosyl-5-O-(3′-N-desmethyl-3′-N-2-fluoroethyl)-6-O-methyl-3-oxo-12,11-[oxycarbonyl-(4-(4-(2-amino-pyrimidin-5-yl)-imidazol-1-yl)-butyl)-imino)] -erythromycin A,
11,12-dideoxy-3-O-decladinosyl-5-O-(3′-N-desmethyl-3′-N-ethyl)-6-O-methyl-3-oxo-12,11-[oxycarbonyl-(4-(5-Nitro-indol-1-yl)-butyl)-imino] -erythromycin A,
11,12-dideoxy-3-O-decladinosyl-5-O-(2′-O-benzoyl-3′-N-desmethyl-3′-N-isopropyl)-6-O-methyl-3-oxo-12, 11-[oxycarbonyl-(3-(4-pyridin-3-yl)-imidazol-1-yl)-propyl)-imino)] -erythromycin A,
11, 12-dideoxy-3-O-decladinosyl-5-O-(3′-N-desmethyl-3′-N-isopropyl)-6-O-methyl-3-oxo-12,11-[oxycarbonyl-(3-(4-pyridin-3-yl)-imidazol-1-yl)-propyl)-imino)] -erythromycin A,
11,12-dideoxy-3-O-decladinosyl-5-O-(3′-N-desmethyl-3′-N-2-fluoroethyl)-6-O-methyl-3-oxo-12,11-[oxycarbonyl-(4-(4-(4-amino-3,5-difluoro-phen-1-yl)-pyrazol-1-yl)-butyl)-imino)] -erythromycin A,
11, 12-dideoxy-3-O- decladinosyl-5-O-(3′-N-desmethyl-3′-N-ethyl)-6-0-methyl-3-oxo-12, 11-[oxycarbonyl-(3-(4-pyridin-3-yl)-imidazol-1-yl)-propyl)-imino)] -erythromycin A,
11, 12-dideoxy-3-O-decladinosyl-5-O-(2′-O-benzoyl-3′-N-desmethyl-3′-N-ethyl)-6-O-methyl-3-oxo-12, 11-[oxycarbonyl-(3-(4-pyridin-3-yl)-imidazol-1-yl)-propyl)-imino)] -erythromycin A,
11,12-dideoxy-3-O-decladinosyl-5-O-(3′-N-desmethyl-3′-N-isopropyl)-6-O-methyl-3-oxo-12,11-[oxycarbonyl-(5-(4-(2-amino-pyrimidin-5-yl)-pyrazol-1-yl)-pentyl)-imino)] -erythromycin A,
11, 12-dideoxy-3-O-decladinosyl-5-O-(2′-O-benzoyl -3′-N-desmethyl-3′-N-isopropyl)-6-O-methyl-3-oxo-12, 11-[oxycarbonyl-(4-(4-pyridin-3-yl)-imidazol-1-yl)-butyl)-imino)] -erythromycin A,
11,12-dideoxy-3-O-decladinosyl-5-O-(3′-N-desmethyl-3′-N-isopropyl)-6-O-methyl-3-oxo-12,11-[oxycarbonyl-(4-(4-pyridin-3-yl)-imidazol-1-yl)-butyl)-imino)] -erythromycin A,
or a pharmaceutically acceptable salt, pharmaceutically acceptable solvate, enantiomer, diastereomer or polymorph thereof.

4. A pharmaceutical composition comprising therapeutically effective amounts of one or more compounds of claim 1 together with one or more pharmaceutically acceptable carriers, excipients, diluents or mixture thereof.

5. A method for treating or preventing a condition caused by or contributed to by bacterial infection comprising administering to a mammal in need thereof therapeutically effective amounts of one or more compounds of claim 1.

6. The method of claim 5, wherein the condition is selected from community acquired pneumonia, upper or lower respiratory tract infections, skin or soft tissue infections, acne vulgaris, hospital acquired lung infections, hospital acquired bone or joint infections, mastitis, catheter infection, foreign body, prosthesis infections, peptic ulcer disease or combination thereof.

7. The method of claim 5, wherein the bacterial infection is caused by gram positive, gram negative or anaerobic bacteria.

8. The method of claim 7, wherein the gram positive, gram negative or anaerobic bacteria is selected from Staphylococci, Streptococci, Enterococci, Haemophilus, Moraxalla spp., Chlamydia spp., Mycoplasin, Legionella spp., Mycobacterium, Helicobacter, Clostridium, Bacteroides, Corynebacterium, Bacillus, Propionibacterium acnes or Enterobactericeae.

9. The method of claim 8, wherein the bacterium is a Staphylococci, Streptococci, or Enterococci.

10. The method of claim 9, wherein the Staphylococci, Streptococci, or Enterococci is drug resistant.

11. A process for preparing a compound of Formula 12, 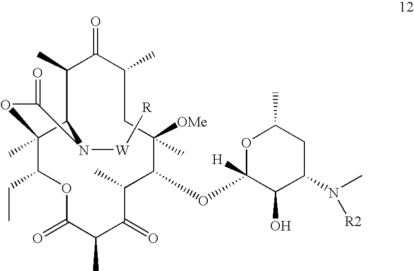
or a pharmaceutically acceptable salt, pharmaceutically acceptable solvate, enantiomer, diastereomer or polymorph thereof, wherein:
R² is C₂-C₆ alkyl, alkenyl, alkynyl, cycloalkyl, aryl, heterocycle, aralkyl, (heterocyclic)alkyl or COR⁴, wherein
R⁴ is hydrogen, alkyl or aralkyl;
W is (CH₂)_{q}, CR⁵R⁶, NR⁵ or SO₂, wherein
q is an integer of from 2 to 10;
R⁵ and R⁶ are independently hydrogen or alkyl;
(CH₂)_{q} is optionally interrupted by one or more of unsaturated bonds, oxygen, sulfur, CO, CS, SO₂, NR₁ or combination thereof; and one or more hydrogen atoms of (CH₂)_{q} group is optionally replaced by halogen, alkyl, hydroxyl or alkoxy, wherein
R₁ is hydrogen, alkyl, alkenyl, alkynyl, COR₂ or (CH₂)ₘR₂;
R₂ is alkyl, alkenyl, alkynyl, aryl or heterocycle;
m is an integer of from 2 to 4R is R₆NCOR₇, aryl or heterocycle; or
R₆ and R₇ are independently aryl or heterocycle;
which method comprises the steps of:
(a) hydrolyzing clarithromycin of Formula 2 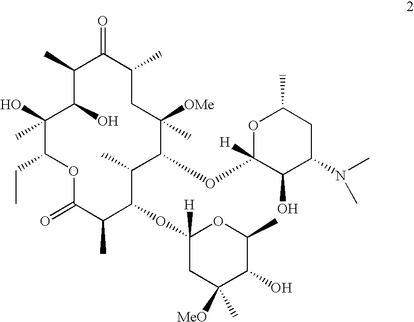
to form a compound of Formula 3 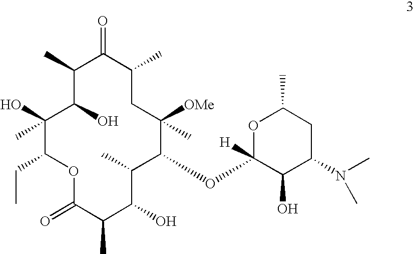
(b) protecting the compound of Formula 3 with one or more reagents of Formula R¹₂O or R¹X (wherein X is halogen) to form a compound of Formula 4 (wherein R¹=COPh) 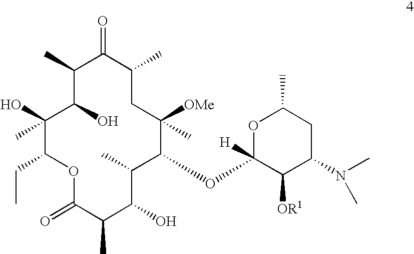
(c) reacting the compound of Formula 4 with one or more reagents selected from triphosgene, ethylene dicarbonate or a mixture thereof to form a compound of Formula 5 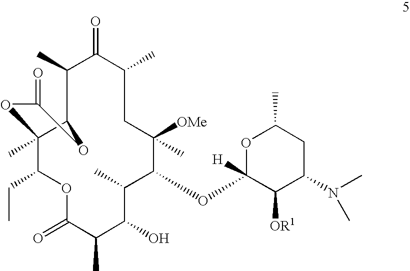
(d) reacting the compound of Formula 5 with one or more organic bases selected from tetramethyl guanidine, trimethylamine or a mixture thereof to form a compound of Formula 6 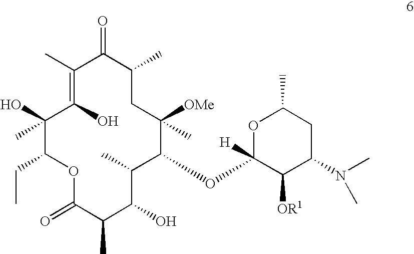
(e) oxidizing the compound of Formula 6 to form a compound of Formula 7 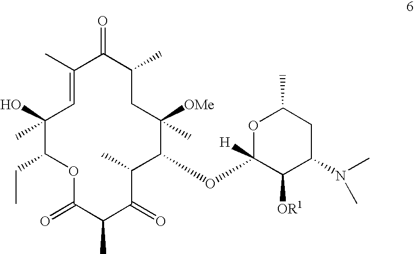
(f) desmethylating the compound of Formula 7 at the 3′-N-dimethyl group to form a compound of Formula 8 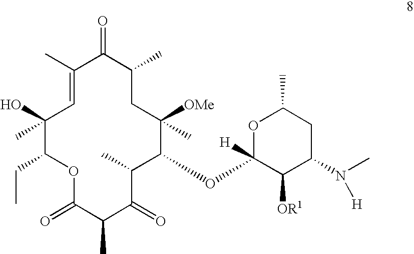
(g) alkylating the compound of Formula 8 with one or more reagents of Formula R²CHO, R²₂CO or R²X (wherein X is halogen) to form a compound of Formula 9 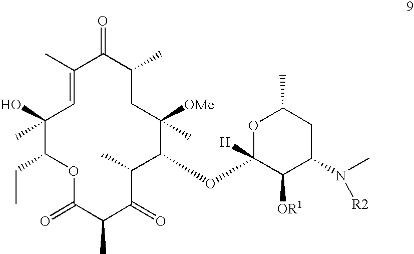
(h) reacting the compound of Formula 9 with N,N′-carbonyldiimidazole to form a compound of Formula 10 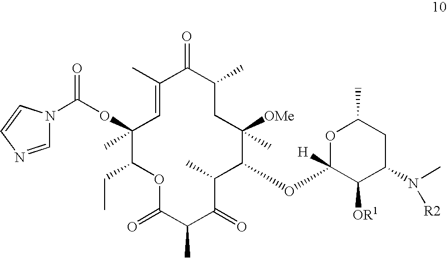
(i) reacting the compound of Formula 10 with a compound of Formula RWNH₂ to form a compound of Formula 11, and 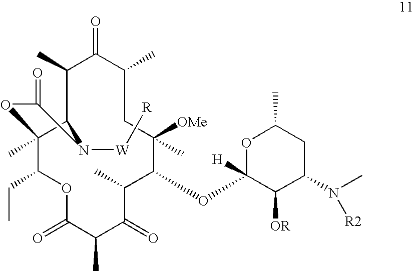
(j) deprotecting the compound of Formula 11 to form a compound of Formula 12.
